(19) United States
(12) Patent Application Publication
(10) Pub. No.: US 2021/0375484 A1
(43) Pub. Date: Dec. 2, 2021

(54) OBJECT-BASED CONTACT TRACING

(71) Applicant: Global Payments Gaming Services Inc., Las Vegas, NV (US)

(72) Inventor: Andrew J. Schwartz, Las Vegas, NV (US)

(73) Assignee: Global Payments Gaming Services Inc., Las Vegas, NV (US)

(21) Appl. No.: 17/333,123

(22) Filed: May 28, 2021

Related U.S. Application Data
(60) Provisional application No. 63/031,104, filed on May 28, 2020.

(51) Int. Cl. G16H 50/80; H04W 4/029

(52) U.S. Cl. CPC G16H 50/80; H04W 4/80; H04W 4/029

(57) ABSTRACT

A contact-tracing (CT) device associated with an inanimate object or location receives a wireless message from a first mobile device indicating that a first user of the first mobile device had previously been exposed to a communicable disease (e.g., Covid). In response, the CT device begins to broadcast tokens indicating that exposure. A second mobile device receiving such a token identifies that exposure to a second user of the second mobile device, thereby enabling the second user to leave the location of the CT device. In some implementations, the CT device informs a backend CT server about the exposure, and the second mobile device queries the CT server about that exposure, thereby enabling the second user to avoid the location of the CT device.

### CROSS-REFERENCE TO RELATED APPLICATIONS

This application claims the benefit of the filing date of U.S. provisional application No. 63/031,104, filed on May 28, 1920 as attorney docket no. 1231.045PROV, the teachings of which are incorporated herein by reference in their entirety.

### BACKGROUND

### Field of the Disclosure

The present disclosure relates to systems for contact tracing for epidemics such as (but not limited to) the Covid-19 crisis.

### Description of the Related Art

This section introduces aspects that may help facilitate a better understanding of the disclosure. Accordingly, the statements of this section are to be read in this light and are not to be understood as admissions about what is prior art or what is not prior art.

Contact tracing is often a key part of effectively dealing with an epidemic, such as the Covid-19 crisis. Companies such as Google and Apple are developing cell phone applications (apps) to support human-to-human contact tracing. According to these apps, if the person has met certain criteria, then that person's cell phone will periodically or intermittently transmit a token, i.e., a wireless, e.g., Bluetooth, message identifying one or more characteristics of that person. Each other cell phone that is within range to receive that token will process the token in an appropriate manner.

For example, if a cell phone user has previously tested positive for the Covid-19 virus, then that information is programmed into the cell phone app and the user's cell phone will transmit tokens indicating that the tokens were sent from a cell phone whose user previously tested positive for the Covid-19 virus. In response, each other cell phone, also running the app, that receives such a token will notify its user (and/or other parties) in some manner that the user is or was within the proximity of someone with the Covid-19 virus.

### BRIEF DESCRIPTION OF THE DRAWINGS

Embodiments of the disclosure will become more fully apparent from the following detailed description, the appended claims, and the accompanying drawings in which like reference numerals identify similar or identical elements.

FIG. 1 is a block diagram of a network according to one embodiment of the invention;

FIG. 2 is a flow diagram of the processing within the network of FIG. 1 according to an example scenario;

FIG. 3 is a flow diagram of the processing within the network of FIG. 1 according to another example scenario; and

FIG. 4 is a generic block diagram of each CT device, the CT server, and each mobile device of FIG. 1 according to different possible implementations.

### DETAILED DESCRIPTION

Detailed illustrative embodiments of the present disclosure are disclosed herein. However, specific structural and functional details disclosed herein are merely representative for purposes of describing example embodiments of the present disclosure. The present disclosure may be embodied in many alternate forms and should not be construed as limited to only the embodiments set forth herein. Further, the terminology used herein is for the purpose of describing particular embodiments only and is not intended to be limiting of example embodiments of the disclosure.

As used herein, the singular forms “a,” “an,” and “the,” are intended to include the plural forms as well, unless the context clearly indicates otherwise. It further will be understood that the terms “comprises,” “comprising,” “includes,” and/or “including,” specify the presence of stated features, steps, or components, but do not preclude the presence or addition of one or more other features, steps, or components. It also should be noted that in some alternative implementations, the functions/acts noted may occur out of the order noted in the figures. For example, two figures shown in succession may in fact be executed substantially concurrently or may sometimes be executed in the reverse order, depending upon the functions/acts involved.

In addition to being carried by humans and being transmitted directly from human to human, the Covid-19 virus can also be transmitted indirectly between humans. For example, if someone with the Covid-19 virus coughs into his hands and then touches an inanimate object without first thoroughly washing his hands, the virus may be transferred to and remain active on the surface of that object for a certain period of time. If, within that period of time, someone else, who is not currently infected by the virus, touches that object and then touches her face without first thoroughly washing her hands, that second person may thereby become infected by the virus, even without ever being within proximity of the infected, first person.

According to certain embodiments of this disclosure, suitable wireless devices, such as Bluetooth dongles, are configured to run a special contact-tracing (CT) app that supports contact tracing between humans and inanimate objects in a manner analogous to the human-to-human contact tracing already envisioned to be provided by cell phone apps. For example, such a CT device could be mounted in, on, or near a coffeemaker in an office. If and when an infected person with his cell phone on him, uses the coffeemaker, the CT device will receive one or more tokens from the infected person's cell phone indicating that an infected person was proximate to the coffeemaker, and the CT device would then transition from an untriggered state to a triggered state. In some implementations, a CT device in the untriggered state (aka an untriggered CT device) does not transmit tokens, and a CT device in the triggered state (aka a triggered CT device) does transmit tokens. As such, the triggered CT device for the coffeemaker would then begin to transmit its own tokens indicating that an infected person is or had been proximate to the coffeemaker. Depending on the particular implementation, the tokens might or might not explicitly identify the coffeemaker as being the inanimate object with which the CT device is associated. When a second person approaches the coffeemaker, that person's cell phone will receive tokens from the coffeemaker's triggered CT device and notify the second person (and/or other parties) that the second person is or was proximate to the triggered CT device of an inanimate object that may have the Covid-19 virus on it. Depending on the implementation, the tokens may identify the coffeemaker explicitly.

Note that, in some implementations, CT devices transmit tokens while in either the untriggered or triggered state, where untriggered CT devices transmit tokens indicating the absence of tokens received from cell phones of anyone with the Covid-19 virus.

When this disclosure describes certain operations performed by a cell phone, it should be understood that those operations are performed by a CT app running on that cell phone. Although this disclosure is described in the context of cell phones, those skilled in the art will understand that the technology can operate with any suitable wireless device, such as (without limitation) a cell phone, a tablet, a laptop computer, a smart watch, a fitness tracker, a smart band, a smart medallion, or a smart key fob. Note that, depending on the particular implementation, the CT app running on cell phones may be the same or different from the CT app running on the CT devices.

Different instances of a CT device of the present disclosure can be provisioned in, on, or near and associated with an almost endless variety of different inanimate objects, such as (without limitation) hotel rooms, restaurants including individual tables and bar counters, public bathrooms including individual stalls, urinals, and sinks, airplanes including individual rows and seats, spas including individual treatment areas, salons and barbers including individual chairs, valet waiting areas, rental cars, taxis, private cars such as those used by Uber and Lyft, rental bicycles, bowling alleys, shoes, and balls, classrooms, dormitory rooms, and stadium/arena seating.

According to certain embodiments, CT devices can be configured to be reset either manually or automatically. For example, if the object associated with a triggered CT device is cleaned by someone, then a wireless reset message can be transmitted, e.g., from that person's cell phone, to instruct the triggered CT device to return to its untriggered state. In addition, since the Covid-19 virus does not live indefinitely on the surface of an inanimate object, in some implementations, a timer is set when a CT device becomes triggered and then, when the timer expires, the triggered CT device will automatically transition back to the untriggered state.

In some implementations, each token includes a timestamp indicating either the time at which the CT device became triggered or the duration of time since the CT device became triggered. In either case, a token-receiving cell phone can determine whether and what type of notification to provide to its user (and/or third parties). For example, the cell phone could determine whether or not to notify the user (and/or third parties) at all. Alternatively or in addition, the cell phone could present the timestamp information to the user (and/or third parties) so that the user (and/or the third parties) can decide how to react.

In some implementations, a user can use his/her cell phone (or other suitable electronic device) to access a special CT website to determine whether remotely located inanimate objects have triggered CT devices. For example, the user may be able to use the CT website to determine whether or not a specific room in a specific hotel in a specific city has a triggered CT device. Similarly, the user may be able to use the CT website to identify only those rooms in the hotel having only untriggered CT devices.

In certain embodiments, the present disclosure is implemented in a distributed network of CT devices and cell phones running CT apps, where the CT devices wirelessly transmit and receive tokens to and from the cell phones via Bluetooth or other suitable relatively short-range communication protocol. In some implementations, the network also includes one or more backend servers, where the CT devices communicate either wirelessly or via wireline with the backend server(s) and/or the cell phones communicate wirelessly with the backend server(s) using conventional cellular communications.

In some implementations, the CT devices transmit tokens that explicitly identify their associated inanimate objects, where the cell phones may receive and pass on that information to the backend server(s). In other implementations, the CT devices may communicate directly with the backend server(s) to identify whether or not they have been triggered.

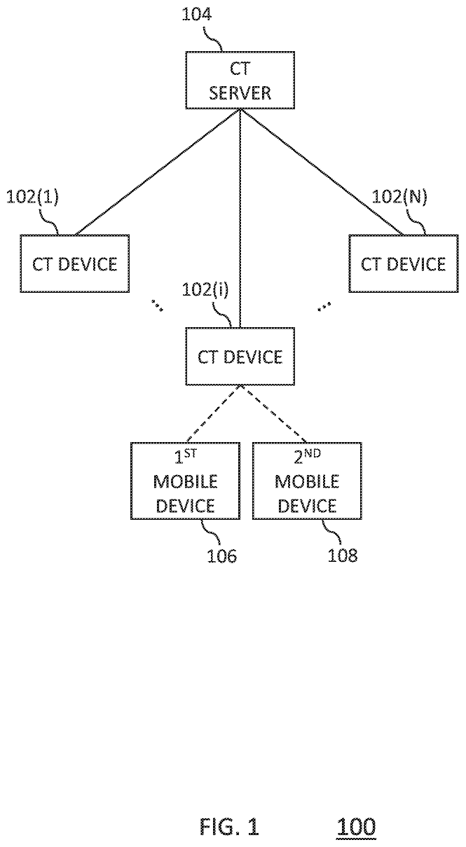

FIG. 1 is a block diagram of a network 100 according to one embodiment of the invention. As shown in FIG. 1, the network 100 includes a number of contact-tracing (CT) wireless devices 102(1)-102(N), each of which is associated with an inanimate object or location, where the CT devices 102 are connected to communicate (either wirelessly or via wireline) with a backend CT server 104. In addition, FIG. 1 shows two mobile wireless devices 106 and 108, where the first mobile device 106 is associated with a first user who has been diagnosed with Covid-19, and the second mobile device 108 is associated with a second user who has not been diagnosed with Covid-19.

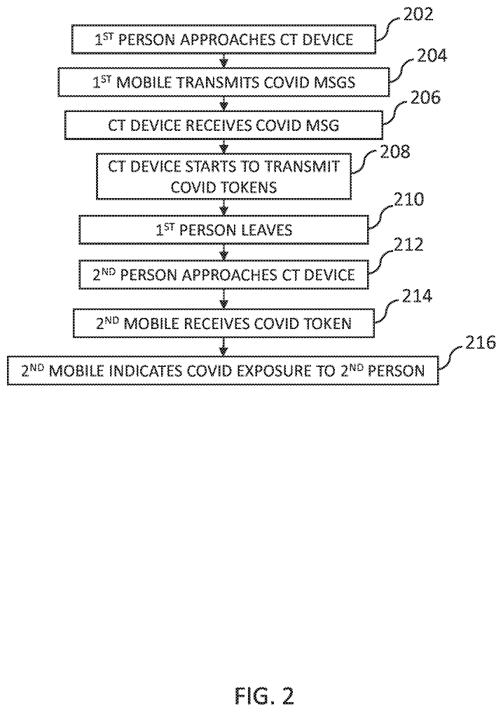

Although both mobile devices 106 and 108 are shown proximate to the same CT device 102(i), in an example scenario described below in the context of FIG. 2, the first user having Covid-19 approaches the CT device 102(i) with the first mobile device 106 and then leaves that location. At a later time, the second user without Covid-19 approaches the CT device 102(i) with the second mobile device 108.

FIG. 2 is a flow diagram of the processing within network 100 of FIG. 1 according to the example scenario mentioned above. In this example scenario, the CT device 102(i) is associated with a coffeemaker in the kitchenette of an office building (all not shown).

In step 202, the first user with the first mobile device 106 approaches the CT device 102(i) to make a cup of coffee using the associated coffeemaker. Because the first user has been diagnosed with Covid, the first mobile device 106 has been previously configured to broadcast periodic wireless messages indicating that the first user has Covid, as shown in step 204.

In step 206, the CT device 102(i) receives one or more of those Covid messages broadcasted from the first mobile device 106. In response, in step 208, the CT device 102(i) starts to broadcast wireless Covid tokens indicating that someone with Covid had previously approached the CT device 102(i). In step 210, the first user leaves the location of the CT device 102(i) taking the first mobile device 106.

Subsequently, in step 212, the second user with the second mobile device 108 approaches the CT device 102(i) to make a cup of coffee using the associated coffeemaker. In step 214, the second mobile device 108 receives one or more of those Covid tokens. In response, in step 216, the second mobile device 108 indicates to the second user that someone having Covid had previously approached the CT device 102(i).

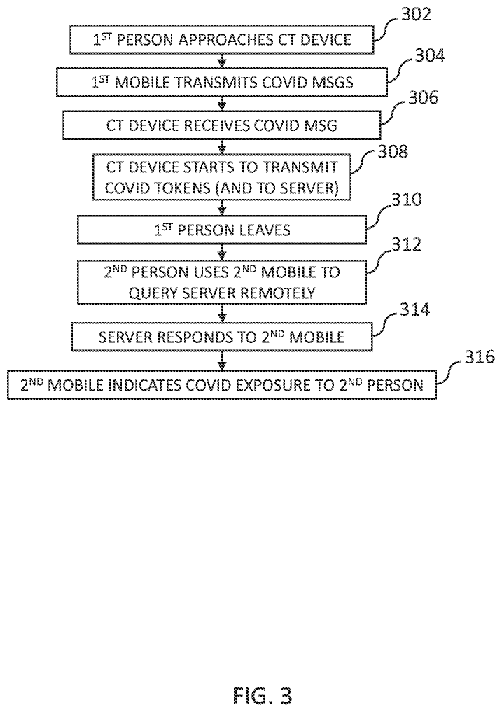

FIG. 3 is a flow diagram of the processing within network 100 of FIG. 1 according to another example scenario that is slightly different from the scenario of FIG. 2. In this example scenario, the second user uses the second mobile device 108 to communicate with the backend CT server 104 before approaching the CT device 102(i).

In particular, steps 302-310 are identical to steps 202-210 of FIG. 2, except that, in step 308, the CT device 102(i) also transmits a Covid token to the CT server 104, which updates its records to reflect that someone with Covid previously approached the CT device 102(i).

In step 312, before approaching the CT device 102(i) while outside of the range of the Covid tokens broadcasted by the CT device 102(i), the second user uses the second mobile device 108 to query the CT server 104 for information about the exposure of nearby coffeemakers to Covid. In response, in step 314, the CT server 104 responds to the query by informing the second mobile device 108 that the CT device 102(i) had previously been exposed to someone with Covid. In step 316, the second mobile device 108 indicates that Covid exposure to the second user, who can then choose to stay away from the associated coffeemaker.

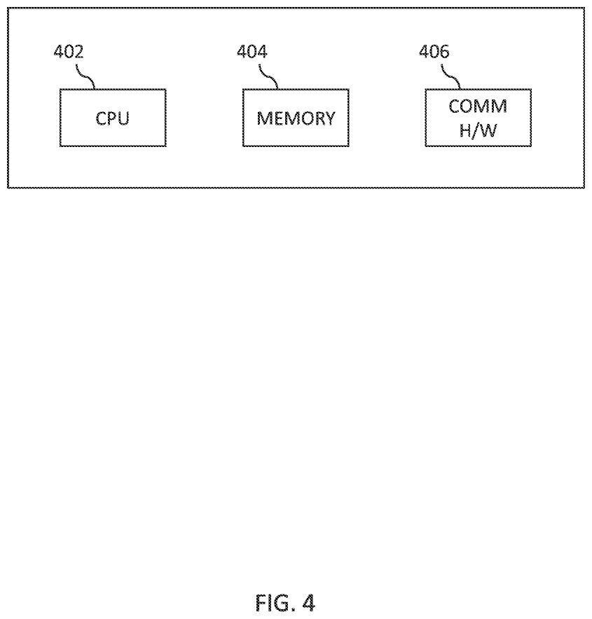

FIG. 4 is a generic block diagram of each CT device 102, the CT server 104, and each mobile device 106, 108 of FIG. 1 according to different possible implementations. As shown in FIG. 4, each device has a processor (e.g., central processing unit (CPU)) 402, memory (e.g., RAM and/or ROM) 404, and communication hardware 406. For each CT device 102, the communication hardware 406 includes a wireless transceiver for communicating with the mobile devices 106, 108 and a server transceiver for communicating with the CT server 104. For the CT server 104, the communication hardware 406 includes a transceiver for communicating with the CT devices 102 and a transceiver for communicating with the mobile devices 106, 108. For each mobile device 106, 108, the communication hardware 406 includes one or more wireless transceivers for communicating with the CT devices 102 and with the CT server 104.

Although the disclosure has been described in the context of the network 100 having a number of different CT devices 102 connected to the backend CT server 104, in other implementations, there is no backend CT server 104 and each different CT device 102 operates independently, as in FIG. 2 and without the capability of FIG. 3.

Although the disclosure has been described in the context of contact tracing for users who have Covid, those skilled in the art will understand that the disclosure can be extended to those who have been exposed to others who have Covid. Thus, the first mobile device 106 may be configured to transmit the same or different types of Covid messages depending on whether the first user has been diagnosed as having Covid herself or whether the first user has been exposed to someone else whose had been diagnosed as having Covid even if she has not been so diagnosed.

Although the disclosure has been described in the context of contact tracing for Covid-19, those skilled in the art will understand that the disclosure can be implemented in the context of any suitable communicable disease.

In certain embodiments, a contact-tracing (CT) device comprises a wireless transceiver configured to communicate with wireless mobile devices and a processor configured to control the wireless transceiver. The wireless transceiver is configured to receive a wireless message from a first mobile device indicating that a first user associated with the first mobile device had previously been exposed to a communicable disease, and the processor is configured to control the wireless transceiver to transmit one or more wireless tokens indicating that a person who had previously been exposed to the communicable disease had been proximate to the CT device.

In at least some of the above embodiments, the CT device further comprises a server transceiver configured to communicate with a CT server to inform the CT server that the person who had previously been exposed to the communicable disease had been proximate to the CT device.

In at least some of the above embodiments, each wireless token indicates a time associated with receipt of the wireless message from the first mobile device.

In at least some of the above embodiments, the processor is configure to maintain a timer associated with the person being proximate to the CT device and stop transmitting the wireless tokens after expiration of the timer.

In certain other embodiments, a mobile device comprises a wireless transceiver configured to communicate with CT devices and a processor configured to control the wireless transceiver. The processor is configured to control the wireless transceiver to transmit a wireless message indicating that a user associated with the mobile device had previously been exposed to a communicable disease, and, upon receipt by the wireless transceiver of a wireless token from a CT device indicating that a person who had previously been exposed to the communicable disease had been proximate to the CT device, the processor is configured to indicate to the user that the CT device had previously been proximate to the person.

In at least some of the above embodiments, the processor is configured to control the wireless transceiver to communicate with a CT server to query the CT server about whether one or more CT devices had previously been proximate to the person.

In at least some of the above embodiments, each wireless token indicates a time associated with receipt of the wireless message, and the processor is configured to determine, based on the time, whether or not to indicate to the user that the CT device had previously been proximate to the person.

In certain other embodiments, a CT server comprises at least one transceiver configured to communicate with at least one CT device and at least one mobile device and a processor configured to control the at least one transceiver. The at least one transceiver is configured to receive information about a CT device having been proximate to a person who had previously been exposed to a communicable disease, and, upon receipt by the at least one transceiver of a query from a mobile device about the CT device having been proximate to the person, the processor is configured to control the at least one transceiver to inform the mobile device about the proximity of the CT device to the person.

Embodiments of the disclosure may be implemented as (analog, digital, or a hybrid of both analog and digital) circuit-based processes, including possible implementation as a single integrated circuit (such as an ASIC or an FPGA), a multi-chip module, a single card, or a multi-card circuit pack. As would be apparent to one skilled in the art, various functions of circuit elements may also be implemented as processing blocks in a software program. Such software may be employed in, for example, a digital signal processor, micro-controller, general-purpose computer, or other processor.

As will be appreciated by one of ordinary skill in the art, the present disclosure may be embodied as an apparatus (including, for example, a system, a machine, a device, a computer program product, and/or the like), as a method (including, for example, a business process, a computer-implemented process, and/or the like), or as any combination of the foregoing. Accordingly, embodiments of the present disclosure may take the form of an entirely software-based embodiment (including firmware, resident software, micro-code, and the like), an entirely hardware embodiment, or an embodiment combining software and hardware aspects that may generally be referred to herein as a “system.”

Embodiments of the disclosure can be manifest in the form of methods and apparatuses for practicing those methods. Embodiments of the disclosure can also be manifest in the form of program code embodied in tangible media, such as magnetic recording media, optical recording media, solid state memory, floppy diskettes, CD-ROMs, hard drives, or any other non-transitory machine-readable storage medium, wherein, when the program code is loaded into and executed by a machine, such as a computer, the machine becomes an apparatus for practicing the disclosure. Embodiments of the disclosure can also be manifest in the form of program code, for example, stored in a non-transitory machine-readable storage medium including being loaded into and/or executed by a machine, wherein, when the program code is loaded into and executed by a machine, such as a computer, the machine becomes an apparatus for practicing the disclosure. When implemented on a general-purpose processor, the program code segments combine with the processor to provide a unique device that operates analogously to specific logic circuits.

Any suitable processor-usable/readable or computer-usable/readable storage medium may be utilized. The storage medium may be (without limitation) an electronic, magnetic, optical, electromagnetic, infrared, or semiconductor system, apparatus, or device. A more-specific, non-exhaustive list of possible storage media include a magnetic tape, a portable computer diskette, a hard disk, a random access memory (RAM), a read-only memory (ROM), an erasable programmable read-only memory (EPROM) or Flash memory, a portable compact disc read-only memory (CD-ROM), an optical storage device, and a magnetic storage device. Note that the storage medium could even be paper or another suitable medium upon which the program is printed, since the program can be electronically captured via, for instance, optical scanning of the printing, then compiled, interpreted, or otherwise processed in a suitable manner including but not limited to optical character recognition, if necessary, and then stored in a processor or computer memory. In the context of this disclosure, a suitable storage medium may be any medium that can contain or store a program for use by or in connection with an instruction execution system, apparatus, or device.

The functions of the various elements shown in the figures, including any functional blocks labeled as “processors,” may be provided through the use of dedicated hardware as well as hardware capable of executing software in association with appropriate software. When provided by a processor, the functions may be provided by a single dedicated processor, by a single shared processor, or by a plurality of individual processors, some of which may be shared. Moreover, explicit use of the term “processor” or “controller” should not be construed to refer exclusively to hardware capable of executing software, and may implicitly include, without limitation, digital signal processor (DSP) hardware, network processor, application specific integrated circuit (ASIC), field programmable gate array (FPGA), read only memory (ROM) for storing software, random access memory (RAM), and non-volatile storage. Other hardware, conventional and/or custom, may also be included. Similarly, any switches shown in the figures are conceptual only. Their function may be carried out through the operation of program logic, through dedicated logic, through the interaction of program control and dedicated logic, or even manually, the particular technique being selectable by the implementer as more specifically understood from the context.

It should be appreciated by those of ordinary skill in the art that any block diagrams herein represent conceptual views of illustrative circuitry embodying the principles of the disclosure. Similarly, it will be appreciated that any flow charts, flow diagrams, state transition diagrams, pseudo code, and the like represent various processes which may be substantially represented in computer readable medium and so executed by a computer or processor, whether or not such computer or processor is explicitly shown.

Unless explicitly stated otherwise, each numerical value and range should be interpreted as being approximate as if the word “about” or “approximately” preceded the value or range.

It will be further understood that various changes in the details, materials, and arrangements of the parts which have been described and illustrated in order to explain embodiments of this disclosure may be made by those skilled in the art without departing from embodiments of the disclosure encompassed by the following claims.

In this specification including any claims, the term “each” may be used to refer to one or more specified characteristics of a plurality of previously recited elements or steps. When used with the open-ended term “comprising,” the recitation of the term “each” does not exclude additional, unrecited elements or steps. Thus, it will be understood that an apparatus may have additional, unrecited elements and a method may have additional, unrecited steps, where the additional, unrecited elements or steps do not have the one or more specified characteristics.

The use of figure numbers and/or figure reference labels in the claims is intended to identify one or more possible embodiments of the claimed subject matter in order to facilitate the interpretation of the claims. Such use is not to be construed as necessarily limiting the scope of those claims to the embodiments shown in the corresponding figures.

It should be understood that the steps of the exemplary methods set forth herein are not necessarily required to be performed in the order described, and the order of the steps of such methods should be understood to be merely exemplary. Likewise, additional steps may be included in such methods, and certain steps may be omitted or combined, in methods consistent with various embodiments of the disclosure.

Although the elements in the following method claims, if any, are recited in a particular sequence with corresponding labeling, unless the claim recitations otherwise imply a particular sequence for implementing some or all of those elements, those elements are not necessarily intended to be limited to being implemented in that particular sequence.

All documents mentioned herein are hereby incorporated by reference in their entirety or alternatively to provide the disclosure for which they were specifically relied upon.

Reference herein to “one embodiment” or “an embodiment” means that a particular feature, structure, or characteristic described in connection with the embodiment can be included in at least one embodiment of the disclosure. The appearances of the phrase “in one embodiment” in various places in the specification are not necessarily all referring to the same embodiment, nor are separate or alternative embodiments necessarily mutually exclusive of other embodiments. The same applies to the term “implementation.”

The embodiments covered by the claims in this application are limited to embodiments that (1) are enabled by this specification and (2) correspond to statutory subject matter. Non-enabled embodiments and embodiments that correspond to non-statutory subject matter are explicitly disclaimed even if they fall within the scope of the claims.

As used in this application, the term “circuitry” may refer to one or more or all of the following: (a) hardware-only circuit implementations (such as implementations in only analog and/or digital circuitry); (b) combinations of hardware circuits and software, such as (as applicable): (i) a combination of analog and/or digital hardware circuit(s) with software/firmware and (ii) any portions of hardware processor(s) with software (including digital signal processor(s)), software, and memory(ies) that work together to cause an apparatus, such as a mobile phone or server, to perform various functions); and (c) hardware circuit(s) and or processor(s), such as a microprocessor(s) or a portion of a microprocessor(s), that requires software (e.g., firmware) for operation, but the software may not be present when it is not needed for operation.” This definition of circuitry applies to all uses of this term in this application, including in any claims. As a further example, as used in this application, the term circuitry also covers an implementation of merely a hardware circuit or processor (or multiple processors) or portion of a hardware circuit or processor and its (or their) accompanying software and/or firmware. The term circuitry also covers, for example and if applicable to the particular claim element, a baseband integrated circuit or processor integrated circuit for a mobile device or a similar integrated circuit in server, a cellular network device, or other computing or network device.

As used herein and in the claims, the term “provide” with respect to an apparatus or with respect to a system, device, or component encompasses designing or fabricating the apparatus, system, device, or component; causing the apparatus, system, device, or component to be designed or fabricated; and/or obtaining the apparatus, system, device, or component by purchase, lease, rental, or other contractual arrangement.

Unless otherwise specified herein, the use of the ordinal adjectives “first,” “second,” “third,” etc., to refer to an object of a plurality of like objects merely indicates that different instances of such like objects are being referred to, and is not intended to imply that the like objects so referred-to have to be in a corresponding order or sequence, either temporally, spatially, in ranking, or in any other manner.

What is claimed is:

1. A contact-tracing (CT) device comprising:
a wireless transceiver configured to communicate with wireless mobile devices; and
a processor configured to control the wireless transceiver, wherein:
the wireless transceiver is configured to receive a wireless message from a first mobile device indicating that a first user associated with the first mobile device had previously been exposed to a communicable disease; and
the processor is configured to control the wireless transceiver to transmit one or more wireless tokens indicating that a person who had previously been exposed to the communicable disease had been proximate to the CT device.

2. The CT device of claim 1, further comprising a server transceiver configured to communicate with a CT server to inform the CT server that the person who had previously been exposed to the communicable disease had been proximate to the CT device.

3. The CT device of claim 1, wherein each wireless token indicates a time associated with receipt of the wireless message from the first mobile device.

4. The CT device of claim 1, wherein the processor is configure to:
maintain a timer associated with the person being proximate to the CT device; and
stop transmitting the wireless tokens after expiration of the timer.

5. A mobile device comprising:
a wireless transceiver configured to communicate with CT devices; and
a processor configured to control the wireless transceiver, wherein:
the processor is configured to control the wireless transceiver to transmit a wireless message indicating that a user associated with the mobile device had previously been exposed to a communicable disease; and
upon receipt by the wireless transceiver of a wireless token from a CT device indicating that a person who had previously been exposed to the communicable disease had been proximate to the CT device, the processor is configured to indicate to the user that the CT device had previously been proximate to the person.

6. The mobile device of claim 5, wherein the processor is configured to control the wireless transceiver to communicate with a CT server to query the CT server about whether one or more CT devices had previously been proximate to the person.

7. The wireless device of claim 5, wherein:
each wireless token indicates a time associated with receipt of the wireless message; and
the processor is configured to determine, based on the time, whether or not to indicate to the user that the CT device had previously been proximate to the person.

8. A CT server comprising:
at least one transceiver configured to communicate with at least one CT device and at least one mobile device; and
a processor configured to control the at least one transceiver, wherein:
the at least one transceiver is configured to receive information about a CT device having been proximate to a person who had previously been exposed to a communicable disease; and
upon receipt by the at least one transceiver of a query from a mobile device about the CT device having been proximate to the person, the processor is configured to control the at least one transceiver to inform the mobile device about the proximity of the CT device to the person.
